(19) United States
(12) Patent Application Publication
(10) Pub. No.: US 2021/0369837 A1
(43) Pub. Date: Dec. 2, 2021

(54) METHODS OF TREATING TIM-3 ELEVATION

(71) Applicant: ADVANTAGENE, INC., Auburndale, MA (US)

(72) Inventors: Estuardo Aguilar-Cordova, Waban, MA (US); Laura Kaye Aguilar, Waban, MA (US); Antonio E. Chiocca, Weston, MA (US); Brian Guzik, Arlington, MA (US); Sean Lawler, Scituate, MA (US); Maria Carmella Speranza, Auburndale, MA (US)

(73) Assignee: CANDEL THERAPEUTICS, INC., Needham, MA (US)

(21) Appl. No.: 16/336,207

(22) PCT Filed: Sep. 26, 2017
(86) PCT No.: PCT/US17/53496

§ 371 Date: Mar. 25, 2019

Related U.S. Application Data
(60) Provisional application No. 62/399,976, filed on Sep. 26, 2016.

(51) Int. Cl. A61K 39/245; A61K 31/522; A61K 31/52; A61K 39/395; A61K 38/21; A61K 38/19; A61K 38/20; A61K 39/39; A61K 35/761; A61K 35/763; A61K 35/76; A61P 35/00

(52) U.S. Cl. CPC A61K 39/245; A61K 2039/55561; A61K 31/52; A61K 39/3955; A61K 38/21; A61K 38/193; A61K 38/2013; A61K 38/2046; A61K 38/208; A61K 38/2086; A61K 38/20; A61K 39/39; A61K 35/761; A61K 35/763; A61K 35/76; A61P 35/00; A61K 31/522

(57) ABSTRACT

The present disclosure provides formulations and therapies for treating subjects with elevated levels of TIM-3 using gene-based cytotoxic immunostimulant therapy alone or with other immunotherapies.

### RELATED APPLICATION

This application claims the benefit of U.S. Provisional Application Ser. No. 62/399,976, filed Sep. 26, 2016, the contents of which is hereby incorporated by reference in its entirety.

### FIELD OF THE INVENTION

The present invention relates to oncology and gene therapy. More specifically the present invention relates to technologies for treating decreasing TIM-3 effects in patients.

### BACKGROUND

It is estimated that more than 1.6 million people in the U.S. will develop cancer in 2016. The most common cancers include breast cancer, lung and bronchus cancer, prostate cancer, colon and rectum cancer, bladder cancer, melanoma, non-Hodgkin lymphoma, thyroid cancer, kidney cancer, leukemia, endometrial cancer and pancreatic cancer. The incidence of cancer is approximately 454 cases per 100,000 men and women per year. The number of deaths is approximately 171 per 100,000 men and women per year. The National Cancer Institute (www.cancer.gov) estimates that nearly 14.5 million people in the U.S. were living with cancer, and that by the year 2024 the number will reach about 19 million (see, www.cancer.gov). Approximately 40% of men and women will be diagnosed with cancer at some point during their lifetime. The majority of deaths from cancer are due to the systemic effects of metastatic disease once the tumor has acquired the ability to grow at a site distant from its origin.

Treatment varies depending on the type of cancer (see, e.g., www.cancer.org). Current therapies for cancer include, for example, surgery, radiation, chemotherapy, immunotherapy, targeted therapy and hormone therapy.

Use of surgical therapies is dependent on the type of cancer and location as well as the stage of disease. For localized cancers, surgical removal is efficacious when organ function is either not indispensable or will not he adversely affected. Surgery may also be performed to reduce the bulk of a tumor and for palliative purposes.

Radiation therapy is used in approximately 50% of the patients that have cancer and may he used to cure the cancer, slow its growth or shrink the size of a tumor. It is often used in junction with surgery and chemotherapy. Some cancers that may, in some circumstances be cured by radiation alone include prostate cancer, head and neck cancer, cervical cancer and brain tumors.

Chemotherapy works by interfering with different phases of the cell cycle or intercalating with the DNA of the cancer cell. As with other therapeutic modalities, it can be used with curative intent, along with radiation, or as a palliative measure. The regimen of chemotherapy used may depend on disease location and tumor pathology. Because chemotherapy is given systemically, and acts on cells throughout the body, the side effects can be more widespread. Immunotherapy uses a patient's immune system to treat cancer. Immunotherapies include monoclonal antibodies, adoptive cell transfer, cytokines, vaccines and Bacillus Calmette-Guerin (BCG).

Such standard cancer therapies each have significant limitations. Seldom are they 100% curative and most have significant associated toxicities. Surgery and radiation are limited in that they only treat locally or loco-regional disease. Also, radiation dose must be limited to prevent damage to normal surrounding tissue. Chemotherapy affects all tissues of the body since it is given systemically. Different chemo-therapeutic agents affect different organs differently. A common side effect, and one that often limits the dose amount and duration of chemotherapy, is depression of white blood cell counts due to its suppressive effects on bone marrow.

Unfortunately, the ability of the immune system to halt or delay cancer does not apply to all tumor types, and not all patients respond to immune-based therapies. Some approaches to stimulate the immune system and target tumor cells, include monoclonal antibodies to target antigens found on cancer cells, immune checkpoint inhibitors to disinhibit an immune response against tumor cells, cancer vaccines and gene based immune stimulants. To date, the use of gene-based immune stimulants in the treatment of cancers has shown promise in some patients and in some tumor types. Unfortunately, the ability of the immune system to halt or delay cancer does not apply to all tumor types, and not all patients respond to immune-based therapies.

Thus improved therapies are needed to increase the percentage of patients that benefit from these therapies by successfully treating and extending the overall survival of cancer patients.

### SUMMARY

It has been discovered that administration of a virus-based immune stimulant and an anti-herpetic pro-drug reduces the levels of TIM-3 in subjects suffering from cancer.

This discovery has been exploited to provide the present disclosure, which, in part, comprises methods of inhibiting TIM-3-mediated down-regulation of effector T cells function in a subject having an immune response to a tumor, comprising treating; the subject with a therapeutically effective amount of a gene-mediated cytotoxic immunostimulant (GNUS) such that effector T cell function is up-regulated and tumor burden is reduced.

In some embodiments, the GMIS therapy comprises administering an oligonucleotide-based cytotoxic immune stimulant and a prodrug.

In certain embodiments, the oligonucleotide-based cytotoxic immune stimulant comprises a virus-based immune stimulant. In particular embodiments, the oligonucleotide-based cytotoxic immune stimulant comprises a gene-based immune stimulant. In some embodiments, in the oligonucleotide-based cytotoxic immune stimulant comprises an adenoviral vector, an adeno-associated viral (AAV) vector, a Herpes viral vector, a vaccinia viral vector, a retroviral vector, or lentiviral vector. In certain embodiments, the oligonucleotide-based cytotoxic immune stimulant comprises an adenovirus-mediated Hepes simplex virus thymidine kinase (AdV-tk) or cytosine deamidase (CD). In some embodiments, AdV-tk comprises aglatimagene besadenovec.

In some embodiments, the prodrug comprises an anti-herpetic pro-drug. On particular embodiments, the anti-herpetic pro-drug comprises ganciclovir, valaciclovir, acyclovir, famciclovir, pemcyclovir, analogs thereof or a combination thereof.

In certain embodiments, the prodrug and the oligonucleotide-based immune stimulant are administered concurrently or serially. In some embodiments, the prodrug is administered after administration of the oligonucleotide-based cytotoxic immune stimulant. In particular embodiments, the prodrug is administered for at least 1 day after administration of the oligonucleotide-based cytotoxic immune stimulant. In other embodiments, the prodrug is administered before administration of the oligonucleotide-based cytotoxic immune stimulant.

In some embodiments, the pro-drug is administered orally, intraperitoneally, intrathecally, intravenously, intravitreously, intralesionally, intratumorally, or intrapleurally.

In some embodiments, the subject being treated has also been treated or is being treated with an additional therapy that up-regulates TIM-3 expression. In certain embodiments, the additional therapy comprises immune checkpoint inhibitor therapy, cytokine mediated therapy, treatment with an immune activation-stimulating adjuvant, and/or treatment with a tumor-associated antigen.

Where the additional therapy comprises administration of an immune checkpoint inhibitor, the immune checkpoint inhibitor can comprise an anti-PD-1 inhibitor, an anti-PDL-1 inhibitor, an anti-CTLA-4 inhibitor, or a combination thereof. In some embodiments, the immune checkpoint inhibitor comprises an antibody, such as an anti-PD-1 antibody. In certain embodiments, the anti-PD-1 antibody comprises pembrolizumab, nivolumab, analogs thereof, or mixtures thereof. In other embodiments, the checkpoint inhibitor comprises an anti-PDL-1 antibody, such as, but not limited to, durvalumab, Atezolizumab, Avelumab, analogs thereof, or combinations thereof.

In still other embodiments, the immune checkpoint inhibitor comprises an anti-CTLA-4 antibody such as, but not limited to, ipilimumab, tremelimumab, MDX-010, analogs thereof, or combinations thereof.

In another embodiment, the additional therapy comprises a cytokine-mediated therapy. In some embodiment, the cytokine-mediated therapy comprises administration of a therapeutically effective amount of IL-2, IL-7, IL-I2, IL-15, IL-18, IL-21, IL-27, GM-CSF, FLT-3, Interferon, or combinations thereof.

In yet other embodiments, the additional therapy comprises administration of an immune adjuvant such as, but not limited to, a Toll-like Receptor agonist. In particular embodiments, the immune adjuvant comprises CpG or GLA.

In still other embodiments, the additional therapy comprises administration of a tumor-associated antigen. In sonic embodiments, the tumor-associated antigen is in a vaccine. In certain embodiments, the vaccine comprises a replicating or non-replicating microbial vector which encodes the tumor-associated antigen. In particular embodiments, vector is a viral or bacterial vector.

In some embodiment, the subject being treated is suffering from, or susceptible to, a cancer. In certain embodiments, the cancer is malignant pleural effusion, lung cancer, mesothelioma, colon cancer, prostate cancer, breast cancer, skin cancer, liver cancer, bone cancer, pancreas cancer, ovary cancer, testis cancer, bladder cancer, kidney cancer, brain cancer, head cancer, or neck cancer.

In some embodiments, the immune response in the subject is increased upon implementation of the method of treatment.

In another aspect, the disclosure provides a method of inhibiting TIM-3-mediated down-regulation of an immune response in a subject, comprising treating the subject with a therapeutically effective amount of a gene-mediated cytotoxic immunostimulant (GMIS) such that effector T cell function is up-regulated and the immune response is up-regulated/increased.

### BRIEF DESCRIPTION OF THE DRAWING

The foregoing and other objects of the present disclosure, the various features thereof, as well as the disclosure itself may be more fully understood from the following description, when read together with the accompanying drawings in which:

FIG. 1 is a graphic representation depicting the exemplary cytotoxic effect in glioma cells (GL261, Mut3 and U251) untreated or treated with ganciclovir (GCV), an adenovirus-mediated Herpes simplex virus thymidine kinase (AdV-tk) or the combination of GCV and AdV-tk (e.g., gene mediated cytotoxic immunotherapy (GMCI));

FIG. 2A is a graphic representation depicting the exemplary quantification of immunocytochemical detection of histone H2AX phosphorylated on Ser-139 in GL261, Mut3, and U251 untreated cells or cells treated with GCV or adenovirus-mediated Herpes simplex virus thymidine kinase (AdV-tk) alone or in combination with GCV;

FIG. 2B are representations of confocal microscope images depicting the immunocytochemical detection of histone H2AX phosphorylated on Ser-139 in GL261, Mut3, and U251 untreated cells or cells treated with GCV or adenovirus-mediated Herpes simplex virus thymidine kinase (AdV-tk) alone or in combination with GCV;

FIG. 3A is a representation of a flow cytometric analysis of the detection of cell surface PD-L1 in human gliomna stem-like cells (GSC) mock treated (mock) or GMCI treated (GMCI), or analysis cells stained with irrelevant antibody (isotype control);

FIG. 3B is a graphic representation of the quantification by flow cytometry of cell surface PD-L1 expression in four human GSCs either mock treated or treated with GMCI;

FIG. 3C are representations of conthcal micrographs depicting the exemplary detection of cell surface PD-L1 by immunofluorescent staining of PD-L1 in untreated and GMCI treated GL261-bearing mice, where nuclei were stained with Hocscht (left) tumor cells stained for vimentin (center) and PD-L1 (right);

FIG. 4A is a graphic representation showing the detection of cell surface PD-L1 in macrophages from tumor-bearing mice treated in vivo with mock treatment or GMCI treatment:

FIG. 4B is a graphic representation showing the detection of cell surface PD-L1 in microglial cells from tumor-bearing mice treated in vivo with mock treatment or GMCI treatment;

FIG. 5A is a graphic representation depicting the exemplary detection of INF-β release from CT2A cells, either mock treated or treated with GCV, AdV-tk, or Ad V-tk+GCV (GMCI);

FIG. 5B is a graphic representation depicting the exemplary detection of INF-β release from GL261 cells, either mock treated or treated with GCV, AdV-tk, or AdV-tk+GCV (GMCI);

FIG. 5C is a graphic representation depicting the exemplary detection of PD-L1 protein in the CT2A cell line without specific antibody (isotype) or with specific antibody without treatment (untreated), or treated with INFα or treated with INFβ;

FIG. 5D is a graphic representation depicting the exemplary quantification of the expression of PD-L1 protein in CT2A cell line without treatment (untreated), or treated with INFα or with INFβ;

FIG. 5E is a graphic representation depicting the exemplary detection of PD-L1 protein in the GL261 cell line without specific antibody (isotype) or with specific antibody without treatment (untreated), or treated with INFα, or treated with INFβ;

FIG. 5F is a graphic representation depicting the exemplary quantification of the expression of PD-L1 protein in the GL261 cell line without treatment (untreated), or treated with INFα or treated with INFβ;

FIG. 6 is a presentation of an immunoblot depicting the exemplary expression of the cGAS gene in the GL261 and CT2A cells either untreated, treated with GCV, treated with AdV-tk, related with a combination of GCV and AdV-tk, where GAPDH expression is a control;

FIG. 7A is a schematic representation of an exemplary protocol where mice were either untreated, treated with AdV-tk and gancyclovir (GCV), treated with anti-PD1 (aPD-1), or treated with both AdV-tk and GCV and aPD-1;

FIG. 7B is a graphic representation depicting the exemplary percent survival of mice were administered GL261-Luc2 glioma cells followed by treatment with either AdV-tk with GCV (GMCI), anti-PD-1 (e.g., “aPD-1”) antibody, or the combination of GMCI and aPD-1 (e.g., “Combo”), the untreated mice did not receive any treatment;

FIG. 7C is a series of representations of bioluminescent images depicting the exemplary detection at Day 21 of the tumor burden in mice that were administered GL261-Luc2 glioma cells followed by treatment with either AdV-tk with GCV (GMCI), anti-PD-1 (e.g., “aPD-1”) antibody, or the combination of GMCI and aPD-1 (e.g., “Combo”), where the untreated mice did not receive any treatment;

FIG. 7D is a graphic representation depicting the exemplary percent survival of mice that had been challenged with GL261-Luc2 cells after being designated as Long Term Survivors (LTS) in the protocol described in FIG. 7A, or were age matched controls naive to GL261 cells (Untreated);

FIG. 7E is a series of representations of bioluminescent images depicting the exemplary detection at Day 21 of the tumor burden in mice that had been challenged with GL261-Luc2 cells after being designated as Long Term Survivors (LTS) in the protocol described in FIG. 7A, or were age matched controls naive to GL261 cells (Untreated);

FIG. 8A is a representation of a scatter plot depicting exemplary quantitation of CD3+ as a percentage of live CD45+ intratumoral cells from mice that were administered GL261 cells and subsequently untreated, treated with GMCI alone, with anti-PD-1 antibody (aPD-1) alone, or with the combination of GMCI and anti-PD-1 antibody (Combo);

FIG. 8B is a representation of a scatter plot depicting the exemplary quantitation of IFN-g+ as a percentage of live CD45+CD3+ intratumoral cells from mice that were administered GL261 cells and subsequently untreated, treated with GMC1 alone, with anti-PD-1 antibody (aPD-1) alone, or with the combination of GMCI and anti-PD-1 antibody (Combo);

FIG. 8C is a representation of a scatter plot depicting the exemplary quantitation of CD8+ effector T cells as a percentage of live CD451-CD3+ intratumoral cells from mice that were administered GL261 cells and subsequently untreated, treated with GMCI alone, with anti-PD-1 antibody (aPD-1) alone, or with the combination of GMCI and anti-PD-1 antibody (Combo);

FIG. 8D are representations of the results of flow cytometry depicting the exemplary detection of CD8+/granzyme B+ effector T cells as a percentage of live CD45+CD3+CD8+ cells intratumoral cells from a mouse that was administered GL261 cells and subsequently untreated, treated with GMCI alone, with anti-PD-1 antibody (aPD-1 ) alone, or with the combination of GMCI and anti-PD-1 antibody (Combo);

FIG. 8E is a representation of a scatter plot depicting the exemplary quantification of CD8+/granzyme B+ effector T cells as a percentage of live CD45+CD3+CD8+ cells intratumoral cells from mice that were administered GL261 cells and subsequently untreated, treated with GMCI alone, with anti-PD-1 antibody (aPD-1) alone, or with the combination of GMCI and anti-PD-1 antibody (Combo);

FIG. 9A is a graphic representation depicting the exemplary quantification of intratumoral CD8+/Treg cell from mice that were administered GL261 cells and subsequently untreated, treated with GMC1 alone, with anti-PD-1 antibody (aPD-1) alone, or with the combination of GMCI and anti-PD-1 antibody (Combo);

FIG. 9B is a representation of a scatter plot depicting the exemplary quantification of TIM3+ cells as a percentage of intratumoral lymphocytes from mice that were administered GL261 cells and subsequently untreated, treated with GMCI alone, with anti-PD-1 antibody (aPD-1) alone, or with the combination of GMCI and anti-PD-1 antibody (Combo);

FIG. 9C is a representation of a scatter plot depicting the exemplary quantification of PDI+TIM3+ cells as a percentage of intratumoral CD8+ lymphocytes from mice that were administered GL261 cells and subsequently untreated, treated with GMC1 alone, with anti-PD-1 antibody (aPD-1) alone, or with the combination of GMCI and anti-PD-1 antibody (Combo);

FIG. 9D is a representation of the results of flow cytometry depicting the exemplary detection of CD8+/CTLA4+ effector T cells as a percentage of live CD45+CD3+CD8+ cells intratumoral cells from a mouse that was administered GL261 cells and subsequently untreated, treated with GMC1 alone, with anti-PD-1 antibody (aPD-1) alone, or with the combination of GMCI and anti-PD-1 antibody (Combo);

FIG. 9E is a representation of a scatter plot depicting the exemplary quantification of CTLA4+ cells as a percentage of intratumoral CD8+ lymphocytes from mice that were administered GL261 cells and subsequently untreated, treated with GMCI alone, with anti-PD-1 antibody (aPD-1) alone, or with the combination of GMCI and anti-PD-1 antibody (Combo);

FIG. 10A is a representation of a micrograph showing the exemplary detection of PD-L1 expression in pancreatic tumor in tissue collected from a patient before AdV-tk injection and 14 days of valacyclovir, where paraffin sections from pre-treatment biopsy of patient case # 1A03 were stained with anti-PD-L1 antibody;

FIG. 10B is a representation of a micrograph showing the exemplary detection of PD-L1 expression pancreatic tumor in tissue collected from a patient after AdV-tk injection and prodrug course, where paraffin sections from post-treatment surgical resection of patient case #1A03 were stained with anti-PD-L1 antibody;

FIG. 10C is a representation of a micrograph showing the exemplary detection of PD-L1 expression pancreatic tumor in tissue collected from a patient before AdV-tk injection and prodrug course, where paraffin sections from pre-treatment biopsy of patient case #2A02 were stained with anti-PD-L1 antibody;

FIG. 10D is a representation of a micrograph showing the exemplary detection of PD-L1 expression pancreatic tumor in tissue collected from a patient after AdV-tk injection and prodrug course. Where paraffin sections from post-treatment surgical resection of patient case #2A02 were stained with anti-PD-L1 antibody;

FIG. 11A is a representation of a scatter plot showing the exemplary detection of CD4 cell infiltration in tumors after AdV-tk injection and prodrug course; and

FIG. 11B is a representation of a scatter plot showing the exemplary detection of CD8 cell infiltration in tumors after AdV-tk injection and prodrug course.

### DETAILED DESCRIPTION

The disclosures of these patents, patent applications, and publications in their entireties are hereby incorporated by reference into this application in order to more filly describe the state of the art as known to those skilled therein as of the date of the invention described and claimed herein. The instant disclosure will govern in the instance that there is any inconsistency between the patents, patent applications, and publications and this disclosure.

Unless defined otherwise, all technical and scientific terms used herein have the same meaning as commonly understood by one of ordinary skill in the art to which this disclosure belongs. The initial definition provided for a group or term herein applies to that group or term throughout the present specification individually or as part of another group, unless otherwise indicated.

### Definitions

As used herein, the term “administration” refers to the administration of a composition to a subject or system. Administration to an animal subject (e.g., to a human) may be by any appropriate route. For example, in some embodiments, administration may be bronchial (including by bronchial instillation), buccal, enteral, interdermal, intra-arterial, intradermal, intragastric, intramedullary, intramuscular, intranasal, intraperitoneal, intrathecal, intravenous, intraventricular, within a specific organ (e.g. intrahepatic), mucosal, nasal, oral, rectal, subcutaneous, sublingual, topical, tracheal (including by intratracheal instillation), transdermal, vaginal and vitreal. In some embodiments, administration may be intratumoral or peritumoral. In some embodiments, administration may involve intermittent dosing. In some embodiments, administration may involve continuous dosing (e.g., perfusion) for at least a selected period of time. As is known in the art, antibody therapy is commonly administered parenterally (e.g., by intravenous or subcutaneous injection), “Local administration” refers to administration directly to the site of a lesion, including a tumor, or to the resected site of a tumor, or to a body cavity in which the tumor is or has been located, Local administration may he a bolus injection to the site, or may include a dose delivery vehicle external to, but in contact with the site or implanted at the local site.

The term “agent” as used herein may refer to a compound or entity of any chemical class including, for example, polypeptides, nucleic acids, saccharides, lipids, small molecules, metals, or combinations thereof As will be clear from context, in some embodiments, an agent can be or comprise a cell or organism, or a fraction, extract, or component thereof. An agent may be comprised of, or comprises a natural product in that it is found in and/or is obtained from nature. An agent is or comprises one or more entities that is man-made in that it is designed, engineered, and/or produced through action of the hand of man and/or is not found in nature. An agent may be utilized in isolated or pure form; in some embodiments, an agent may be utilized in crude form. Potential agents are provided as collections orlibraries, for example that may be screened to identify or characterize active agents within them. Some particular agents that may be utilized in accordance with the present invention include, but are not limited to, small molecules, antibodies, active antibody fragments, aptamers, nucleic acids (e.g., siRNAs, shRNAs, DNA/RNA hybrids, antisense oligonucleotides, and ribozymes), peptides, peptide mimetics, etc. An agent may be or may comprise a polymer. Other exemplary agents are not polymers and/or are substantially free of any polymer. An agent may or may not contain at least one polymeric moiety.

The term “regimen” as used herein may refer to the administration of a single agent or multiple agents to a patient in a doses and schedules that has been shown to provide therapeutic benefit. For example, a regimen as used herein includes the administration of an oligonucleotide-based cytotoxic immune stimulant and the administration of a prodrug.

As used herein, the term “antibody” refers to a polypeptide that includes canonical immunoglobulin sequence elements sufficient to confer specific binding to a particular target antigen. For purposes of the present invention, in certain embodiments, any polypeptide or complex of polypeptides that includes sufficient immunoglobulin domain sequences as found in natural antibodies can be referred to and/or used as an “antibody”, whether such polypeptide is naturally produced (e.g., generated by an organism reacting to an antigen), or produced by recombinant engineering, chemical synthesis, or other artificial system or methodology. In some embodiments, an antibody is polyclonal; in some embodiments, an antibody is monoclonal. In some embodiments, an antibody is chimeric and has constant region sequences that are characteristic of mouse, rabbit, primate, or human antibodies. In some embodiments, antibody sequence elements are humanized, primatized, chimeric, etc., as is known in the art. Moreover, the term “antibody” as used herein, can refer in appropriate embodiments (unless otherwise stated or clear from context) to any of the art-known or developed constructs or formats for utilizing antibody structural and functional features in alternative presentation. For example, embodiments, an antibody utilized in accordance with the present invention is in a format selected from, but not limited to, intact IgG, IgE and IgM, bi- or multi-specific antibodies (e.g., Zybodies®, etc.), single chain Fvs, polypeptide-Fc fusions, Fabs, camelid antibodies, masked antibodies (e.g., Probodies®), Small Modular ImmunoPharmaceuticals (“SMIPs™”), single chain or Tandem diabodies (TandAb®), VHHs, Anticalins®, Nanobodies®, minibodies, BiTE®s, ankyrin repeat proteins or DARPINs®, Avimers®, a DART, a TCR-like antibody, Adnectins®, Affilins®, Trans-bodies®, Affibodies®, a TrimerX®, MicroProteins, Fynomers®, Centyrins®, and a KALBITOR®. In some embodiments, an antibody may lack a covalent modification (e.g., attachment of a glycan) that it would have if produced naturally. In some embodiments, an antibody may contain a covalent modification (e.g., attachment of a glycan, a payload [e.g., a detectable moiety, a therapeutic moiety, a catalytic moiety, etc.], or other pendant group [e.g., poly-ethylene glycol, etc.].

As used herein, the term “antibody agent” or “antibody” refers to an agent that specifically binds to a particular antigen. The term encompasses any polypeptide or polypeptide complex that includes immunoglobulin structural elements sufficient to confer specific binding. Exemplary antibody agents include, but are not limited to, human antibodies, primatized antibodies, chimeric antibodies, bi-specific antibodies, humanized antibodies, conjugated antibodies (i.e., antibodies conjugated or fused to other proteins, radiolabels, or cytotoxins), Small Modular ImmunoPharmaceuticals (“SMIPs™”), single chain antibodies, cameloid antibodies, and antibody fragments. As used herein, the term “antibody agent” also includes intact monoclonal antibodies, polyclonal antibodies, single domain antibodies e.g., shark single domain antibodies (e.g., IgNAR or fragments thereof)), multispecific antibodies (e.g. bi-specific antibodies) formed from at least two intact antibodies, and antibody fragments so long as they exhibit the desired biological activity. In some embodiments, the term encompasses stapled peptides. In some embodiments, the term encompasses one or more antibody-like binding peptidomimetics. The term encompasses one or more antibody-like binding scaffold proteins. In come embodiments, the term encompasses monobodies or adnectins. In many embodiments, an antibody agent is or comprises a polypeptide whose amino acid sequence includes one or more structural elements recognized by those skilled in the art as a complementarity determining region (CDR); in some embodiments an antibody agent is or comprises a polypeptide whose amino acid sequence includes at least one CDR (e.g., at least one heavy chain CDR and/or at least one light chain CDR) that is substantially identical to one found in a reference antibody. In some embodiments an included CDR is substantially identical to a reference CDR in that it is either identical in sequence or contains between 1-5 amino acid substitutions as compared with the reference CDR. In some embodiments an included CDR is substantially identical to a reference CDR in that it shows at least 85%, 86%, 87%, 88%, 89%, 90%, 91%, 92%, 93%, 94%, 95%, 96%, 97%, 98%, 99%, or 100% sequence identity with the reference CDR. In some embodiments an included CDR is substantially identical to a reference CDR in that it shows at least 96%, 96%, 97%, 98%, 99%, or 100% sequence identity with the reference CDR. In some embodiments an included CDR is substantially identical to a reference CDR in that at least one amino acid within the included CDR is deleted, added, or substituted as compared with the reference CDR but the included CDR has an amino acid sequence that is otherwise identical with that of the reference CDR. In some embodiments an included CDR is substantially identical to a reference CDR in that 1-5 amino acids within the included CDR are deleted, added, or substituted as compared with the reference CDR but the included CDR has an amino acid sequence that is otherwise identical to the reference CDR. In some embodiments, an included CDR is substantially identical to a reference CDR in that at least one amino acid within the included CDR is substituted as compared with the reference CDR but the included CDR has an amino acid sequence that is otherwise identical with that of the reference CDR. In some embodiments, an included CDR is substantially identical to a reference CDR in that 1-5 amino acids within the included CDR are deleted, added, or substituted as compared with the reference CDR but the included CDR has an amino acid sequence that is otherwise identical to the reference CDR. In some embodiments, an antibody agent is or comprises a polypeptide whose amino acid sequence includes structural elements recognized by those skilled in the art as an immunoglobulin variable domain. In some embodiments, an antibody agent is a polypeptide protein having a binding domain which is homologous or largely homologous to an immunoglobulin-binding domain. In some embodiments, an anti-PD-1 antibody agent is an agent that may interfere with interaction of PD-1 and PD-L1, for example either by disrupting contact or by reducing surface expression level of one or other or both of PD-1 and PD-L1.

Two events or entities are “associated” with one another, as that term is used herein, if the presence, level and/or form of one is correlated with that of the other. For example, a particular entity (e.g., polypeptide, genetic signature, metabolite, etc.) is considered to be associated with a particular disease, disorder, or condition, if its presence, level and/or form correlates with incidence of and/or susceptibility to the disease, disorder, or condition (e.g., across a relevant population). In some embodiments, two or more entities are physically “associated” with one another if they interact, directly or indirectly, so that they are and/or remain in physical proximity with one another. In some embodiments, two or more entities that are physically associated with one another are covalently linked to one another; in some embodiments, two or more entities that are physically associated with one another are not covalently linked to one another but are non-covalently associated, for example by means of hydrogen bonds, van der Waals interaction, hydrophobic interactions, magnetism, and combinations thereof.

As used herein, the term “biological sample” or typically refers to a sample obtained or derived from a biological source (e.g., a tissue or organism or cell culture) of interest, as described herein. In some embodiments, a source of interest comprises an organism, such as an animal or human. In some embodiments, a biological sample is or comprises biological tissue or fluid. In some embodiments, a biological sample may be or comprise bone marrow; blood; blood cells; ascites; tissue or fine needle biopsy samples; cell-containing body fluids; free floating nucleic acids; sputum; saliva; urine; cerebrospinal fluid, peritoneal fluid; pleural fluid; feces; lymph; gynecological fluids; skin swabs; vaginal swabs; oral swabs; nasal swabs; washings or lavages such as a ductal lavages or broncheoalveolar lavages; aspirates; scrapings; bone marrow specimens; tissue biopsy specimens; surgical specimens; feces, other body fluids, secretions, and/or excretions; and/or cells therefrom, etc. In some embodiments, a biological sample is or comprises cells obtained from an individual. In some embodiments, obtained cells are or include cells from an individual from whom the sample is obtained. In some embodiments, a sample is a “primary sample” obtained directly from a source of interest by any appropriate means. For example, in some embodiments, a primary biological sample is obtained by methods selected from the group consisting of biopsy (e.g., fine needle aspiration or tissue biopsy), surgery, collection of body fluid (e.g., blood, lymph, feces etc.), etc. In some embodiments, as will be clear from context, the term “sample” refers to a preparation that is obtained by processing (e.g., by removing one or more components of and/or by adding one or more agents to) a primary sample. For example, filtering using a semi-permeable membrane. Such a “processed sample” may comprise, for example nucleic acids or proteins extracted from a sample or obtained by subjecting a primary sample to techniques such as amplification or reverse transcription of mRNA, isolation and/or purification of certain components, etc.

CTLA-4 (cytotoxic T-lymphocyte-associated protein 4; CTLA4) is an immune checkpoint receptor molecule that down-regulates pathways of T-cell activation. CTLA-4 is also known as CD152 (cluster of differentiation 152). CTLA-4 is constitutively expressed in T_{regs} but only expressed by other cells following activation. Binding of CD80 or CD86 on the surface of antigen-presenting cells to the CTLA-4 receptor turns off, or down regulates T cell response. CTLA-4 is also found in regulatory T cells and contributes to their inhibitory function. Blocking CTLA-4 leads to increase in T cell proliferation and increase in interleukin-2 production.

The terms “cancer”, “malignancy”, “neoplasm”, “tumor”, and “carcinoma”, are used interchangeably herein to refer to cells that exhibit relatively abnormal, uncontrolled, and/or autonomous growth, so that they exhibit an aberrant growth phenotype characterized by a significant loss of control of cell proliferation. In general, cells of interest for detection or treatment in the present application include precancerous (e.g benign), malignant, pre-metastatic, metastatic, and non-metastatic cells. The teachings of the present disclosure may be relevant to any and all cancers. To give but a few, non-limiting examples, in some embodiments, teachings of the present disclosure are applied to one or more cancers such as, for example, hematopoietic cancers including leukemias, lymphomas (Hodgkins and non-Hodgkins), myelomas and myeloproliferative disorders, sarcomas, melanomas, adenomas, carcinomas of solid tissue, squamous cell carcinomas of the mouth, throat, larynx, and lung, liver cancer, genitourinary cancers such as prostate, cervical, bladder, uterine, and endometrial cancer and renal cell carcinomas, bone cancer, pancreatic cancer, skin cancer, cutaneous or intraocular melanoma, cancer of the endocrine system, cancer of the thyroid gland, cancer of the parathyroid gland, head and neck cancers, breast cancer (e.g., gliomas such as n astrocytoma, glioblastoma, oligodendroglioma, ependymoma, mixed glioma, optic glioma or a gliomatosis cerebri), gastrointestinal cancers and nervous system cancers, such as brain cancers, benign lesions such as papillomas, and the like.

As used herein, the term “combination therapy” refers to those situations in which a subject is simultaneously exposed to two or more therapeutic regimens (e.g., two or more therapeutic agents). In some embodiments, the two or more regimens may be administered simultaneously; in some embodiments, such regimens may be administered sequentially (e.g., all “doses” of a first regimen are administered prior to administration of any doses of a second regimen); in some embodiments, such agents are administered in overlapping dosing regimens. In some embodiments, a dosing regimen for one or more agents may comprise a plurality of “cycles” of doses administered according to a specified pattern. In some embodiments, cycles of different agents may be administered serially. In some embodiments, cycles of different agents may he administered concurrently. In some embodiments, “administration” of combination therapy may involve administration of one or more agents or modalities to a subject receiving the other agents or modalities in the combination. For clarity, combination therapy does not require that individual agents be administered together in a single composition (or even necessarily at the same time), although in some embodiments, two or more agents, or active moieties thereof, may be administered together in a combination composition, or even in a combination compound (e.g., as part of a single chemical complex or covalent entity).

As used herein, the term “dosage form” refers to a physically discrete unit of an active agent (e.g., a therapeutic or diagnostic agent) for administration to a subject. Each unit contains a predetermined quantity of active agent. In some embodiments, such quantity is a unit dosage amount (or a whole fraction thereof) appropriate for administration in accordance with a dosing regimen that has been determined to correlate with a desired or beneficial outcome when administered to a relevant population (i.e., with a therapeutic dosing regimen). Those of ordinary skill in the art appreciate that the total amount of a therapeutic composition or agent administered to a particular subject is determined by one or more attending physicians and may involve administration of multiple dosage forms.

As used herein, the term “dosing regimen” refers to a set of unit doses (typically more than one) that are administered individually to a subject, typically separated by periods of time. In some embodiments, a given therapeutic agent has a recommended dosing regimen, which may involve one or more doses. In some embodiments, a dosing regimen comprises a plurality of doses each of which are separated from one another by a time period of the same length; in some embodiments, a dosing regimen comprises a plurality of doses and at least two different time periods separating individual doses. In some embodiments, all doses within a dosing regimen are of the same unit dose amount. In some embodiments, different doses within a dosing regimen are of different amounts. In some embodiments, a dosing regimen comprises a first dose in a first dose amount, followed by one or more additional doses in a second dose amount different from the first dose amount. In some embodiments, a dosing regimen comprises a first dose in a first dose amount, followed by one or more additional doses in a second dose amount same as the first dose amount In some embodiments, a dosing regimen is correlated with a desired or beneficial outcome when administered across a relevant population (i.e., is a therapeutic dosing regimen).

As used herein, the tern “effector function” refers a biochemical event that results from the interaction of an antibody Fe region with an Fc receptor or ligand. Effector functions include but are not limited to antibody-dependent cell-mediated cytotoxicity (ADCC), antibody-dependent cell-mediated phagocytosis (ADCP), and complement mediated cytotoxicity (CMC). In some embodiments, an effector function is one that operates after the binding of an antigen, one that operates independent of antigen binding, or both.

As used herein the term “effector cell” refers to a cell of the immune system that expresses one or more Fe receptors and mediates one or more effector functions. In some embodiments, effector cells may include, but may not he limited to, one or more of monocytes, macrophages, neutrophils, dendritic cells, eosinophils, mast cells, platelets, large granular lymphocytes, Langerhans' cells, natural killer (NK) cells, T-lymphocytes, (Effector T cells), B lymphocytes and may be from any organism including but not limited to humans, mice, rats, rabbits, and monkeys.

As used herein, the term “inumme checkpoint inhibitor” refers to molecules that totally or partially antagonize, reduce, inhibit, interfere with or modulate one or more checkpoint proteins. In some instances, an immune checkpoint inhibitor antagonizes the immune checkpoint protein (e.g., CTLA-4. PD-1 or TIM-3). In other instances, the immune checkpoint inhibitor antagonizes the ligand (e.g., PD-L1, PD-L2, CD80, CD86 and galectin-9) of the immune checkpoint protein. immune checkpoint inhibitors include, for example, small molecules or antibodies and fragments thereof, aptamers, nucleic acids (e.g., siRNAs, shRNAs, DNA/RNA hybrids, antisense oligonucleotides, and ribozymes), peptides, (and peptide mimetics).

The term “PD-1”, refers to Programmed cell death protein 1, and is also known as PD-1 and CD279 (cluster of differentiation 279). This is a cell surface receptor that plays an important role in down-regulating the immune system and promoting self-tolerance by suppressing T cell inflammatory activity. PD-1 is an immune checkpoint and guards against autoimmunity through a dual mechanism of promoting apoptosis in antigen-specific T-cells in lymph nodes while simultaneously reducing apoptosis in regulatory T cells (anti-inflammatory, suppressive T cells).

“PD-L” or “PD-L1” refers to the Programmed death ligand type 1 protein, also known as cluster of differentiation 274 or 87 homolog 1. PD-L1 is a transmembrane protein that may play a major role in suppressing the immune system during particular events such as pregnancy, tissue allografts, autoimmune disease and other disease states such as hepatitis. The binding of PD-L1 to PD-1 or B7.1 transmits an inhibitory signal which reduces the proliferation of the CD8+ T cells at the lymph nodes. In addition, PD-1 is also able to control the accumulation of foreign antigen-specific I cells in the lymph nodes through apoptosis which is further mediated by a lower regulation of the Bcl-2gene.

“PD-1 inhibitors,” refers to the class of drugs that block PD-1, thereby activating the immune system to attack tumors and have been used with varying success to treat some types of cancer.^{[5]}

As used herein, the term “pharmaceutically acceptable” applied to the carrier, diluent, or excipient used to formulate a composition as disclosed herein means that the carrier, diluent, or excipient must be compatible with the other ingredients of the composition and not deleterious to the recipient thereof.

As used herein, the term “pharmaceutical composition” refers to an active or therapeutic agent, formulated together with one or more pharmaceutically acceptable carriers. In some embodiments, active agent is present in unit dose amount appropriate for administration in a therapeutic regimen that shows a statistically significant probability of achieving a predetermined therapeutic effect when administered to a relevant population. In some embodiments, pharmaceutical compositions may be specially formulated for administration in solid or liquid form, including those adapted for the following: oral administration, for example, drenches (aqueous or non-aqueous solutions or suspensions), tablets, e.g., those targeted for buccal, sublingual, and systemic absorption, boluses, powders, granules, pastes for application to the tongue; parenteral administration, for example, by subcutaneous, intramuscular, intravenous or epidural injection as, for example, a sterile solution or suspension, or sustained-release formulation; topical application, for example, as a cream, ointment, or a controlled-release patch or spray applied to the skin, lungs, or oral cavity; intravaginally or intrarectally, for example, as a pessary, cream, or foam; sublingually; ocularly; transdermally; or nasally, pulmonary, and to other mucosal surfaces.

As used herein, a “response to treatment” refers to any beneficial alteration in a subject's condition that occurs as a result of or correlates with treatment. Such alteration may include stabilization of the condition (e.g., prevention of deterioration that would have taken place in the absence of the treatment), amelioration of symptoms of the condition, and/or improvement in the prospects for cure of the condition, etc. It may refer to a subject's response or to a tumor's response. Tumor or subject response may be measured according to a wide variety of criteria, including clinical criteria and objective criteria. Techniques for assessing response include, but are not limited to, clinical examination, positron emission tomatography, chest X-ray CT scan, MRI, ultrasound, endoscopy, laparoscopy, presence or level of tumor markers in a sample obtained from a subject, cytology, and/or histology. Many of these techniques attempt to determine the size of a tumor or otherwise determine the total tumor burden. Methods and guidelines for assessing response to treatment are discussed in Therasse et al. (J. Natl. Cancer Inst. (2000) 92(3):205-216). The exact response criteria can be selected in any appropriate manner, provided that when comparing groups of tumors and/or patients, the groups to be compared are assessed based on the same or comparable criteria for determining response rate. One of ordinary skill in the art will be able to select appropriate criteria.

By “subject” is meant a mammal (e.g., a human, dog, cat, cow, horse, rabbit, pig, etc., including prenatal human forms). In some embodiments, a subject is suffering from a relevant disease, disorder or condition. In some embodiments, a subject is susceptible to a disease, disorder, or condition (e.g., cancer). In some embodiments, a subject displays one or more symptoms or characteristics of a disease, disorder or condition. In some embodiments, a subject does not display any symptom or characteristic of a disease, disorder, or condition. In some embodiments, a subject is someone with one or more features characteristic of susceptibility to or risk of a disease, disorder, or condition. In some embodiments, a subject is a patient. In some embodiments, a subject is an individual to whom diagnosis and/or therapy is and/or has been administered.

As used herein, the phrase “Therapeutic agent” “therapeutic composition”, or “therapy” in general refers to any agent(s) that elicits a desired pharmacological effect when administered to an organism. In some embodiments, an agent is considered to be a therapeutic agent if it demonstrates a statistically significant effect across an appropriate population. In sonic embodiments, the appropriate population may be a population of model organisms. In some embodiments, an appropriate population may be defined by various criteria, such as a certain age group, gender, genetic background, preexisting clinical conditions, etc. In some embodiments, a therapeutic agent is a substance that can be used to alleviate, ameliorate, relieve, inhibit, prevent, delay onset of, reduce severity of, and/or reduce incidence of one or more symptoms or features of a disease, disorder, and/or condition. In some embodiments, a “Therapeutic agent” is an agent that has been or is required to be approved by a government agency before it can be marketed for administration to humans. In some embodiments, a “therapeutic agent” is an agent for which a medical prescription is required for administration to humans.

As used herein, the term “therapeutically effective amount” means an amount that is sufficient, when administered to a population suffering from or susceptible to a disease, disorder, and/or condition in accordance with a therapeutic dosing regimen, to treat the disease, disorder, and/or condition. In some embodiments, a therapeutically effective amount is one that reduces the incidence and/or severity of, stabilizes one or more characteristics of, and/or delays onset of, one or more symptoms of the disease, disorder, and/or condition. Those of ordinary skill in the art will appreciate that the term “therapeutically effective amount” does not in fact require successful treatment be achieved in a particular individual. Rather, a therapeutically effective amount may be that amount that provides a particular desired pharmacological response in a significant number of subjects when administered to patients in need of such treatment. For example, in some embodiments, term “therapeutically effective amount”, refers to an amount which, when administered to an individual in need thereof in the context of inventive therapy, will block, stabilize, attenuate, or reverse a cancer-supportive process occurring in the individual, or will enhance or increase a cancer-suppressive process in the individual. In the context of cancer treatment, a “therapeutically effective amount” is an amount which, when administered to an individual diagnosed with a cancer, will prevent, stabilize, inhibit, or reduce the further development of cancer in the individual. One useful “therapeutically effective amount” of a composition described herein reverses (in a therapeutic treatment) the development of a malignancy such as a brain cancer or helps achieve or prolong remission of a malignancy. A therapeutically effective amount administered to an individual to treat a cancer in that individual may be the same or different from a therapeutically effective amount administered to promote remission or inhibit metastasis. As with most cancer therapies, the therapeutic methods described herein are not to be interpreted as, restricted to, or otherwise limited to a “cure” for cancer; rather the methods of treatment are directed to the use of the described compositions to “treat” a cancer, i.e., to effect a desirable or beneficial change in the health of an individual who has cancer. Such benefits are recognized by skilled healthcare providers in the field of oncology and include, but are not limited to, a stabilization of patient condition, a decrease in tumor size (tumor regression), an improvement in vital functions (e,g., improved function of cancerous tissues or organs), a decrease or inhibition of further metastasis, a decrease in opportunistic infections, an increased survivability, a decrease in pain, improved motor function, improved cognitive function, improved feeling of energy (vitality, decreased malaise), improved feeling of well-being, restoration of normal appetite, restoration of healthy weight gain, and combinations thereof. In addition, regression of a. particular tumor in an individual (e.g, as the result of treatments described herein) may also be assessed by taking samples of tumor cells from the site of a tumor such as a brain tumor (e.g., over the course of treatment) and testing the tumor cells for the level of metabolic and signaling markers to monitor the status of the tumor cells to verify at the molecular level the regression of the tumor cells to a less malignant phenotype. Those of ordinary skill in the art will appreciate that, in some embodiments, a therapeutically effective amount may be formulated and/or administered in a single dose. In some embodiments, a therapeutically effective amount may be formulated and/or administered in a plurality of doses, for example, as part of a dosing regimen.

### TIM-3 Elevated Conditions

The present disclosure provides methods for treating subjects suffering from, or susceptible to, an elevated level of TIM-3.

“TIM-3” or “TIM3” refers to T-cell immunoglobulin and mucin-domain containing-3 protein that is selectively expressed in IFN-γ producing CD4+ T helper and CD8+ cytotoxic T cells. TIM-3 acts as a cell inhibitory receptor and binds the ligand C-type lectin galectin-9. Galectin-9 binding to TIM-3 induces cell death in TIM-3 positive T helper cells and can ameliorate experimental models of autoiminune disease. TIM-3 is also required for induction of tolerance and functions specifically to limit the duration and magnitude of helper and CD8+ T cell response.

Those suffering from the effects of elevated level of TIM-3 that can be treated by the methods of the disclosure are mammals such as humans who are experiencing inflammation or who may also a receiving a therapeutic composition which results in up-regulated tim-3. For example, the subject may or will be receiving immune checkpoint inhibitor therapy, cytokine mediated therapy, immune adjuvants known to stimulate immune activations, or specific tumor-associated antigens including a vaccine that may include replicating or non-replicating microbial vectors. Such patients can be those suffering from a solid tumor or a cancer such as, but not limited to, malignant pleural effusion, lung cancer, methothelioma, colon cancer, prostate cancer, breast cancer, skin cancer, liver cancer, hone cancer, pancreas cancer, ovary cancer, testis cancer, bladder cancer, kidney cancer, brain cancer, head cancer, or neck cancer. For example, the patient may be suffering from brain cancer, such as, but not limited to, a glioma. In particular embodiments, the glioma is an astrocytoma, glioblastoma, oligodendroglioma, ependymoma, mixed glioma, optic glioma or a gliornatosis cerebri.

### Gene-Mediated Cytotoxic Immunostimulant (GMIS) Therapy

The therapies for reducing the level of TIM-3 in a subject according to the disclosure comprise using GMIS therapy. GMIS therapy utilizes an oligonucleotides-based cytotoxic immune stimulant (e.g., genetic bacterial or viral vectors) or other oligonucleotides to deliver therapeutic genes to tumors. GMIS alternatively uses transfection to deliver the genes, themselves, to the subject, and also includes the administration of an anti-herpetic prodrug to the subject.

One useful oligonucleotide-based cytotoxic immune stimulant is a vector such as an adenoviral (AdV) vector. Other useful vectors include Adeno-Associated Virus (AAV) vector, retroviral vectors, and lentiviral vectors. The vector encodes a gene or genes (e.g., thymidine kinase, cytosine deamidase, which are expressed at a tumor site, lesion, area or body cavity before or after tumor resection. Expression of the gene can, for example, make tumor cells susceptible to the cytotoxic effects of a prodrug (e.g., ganciclovir, valacyclovir, and acyclovir). One useful gene expressed by a vector is Herpes Simplex (HSV) thymidine kinase (tk). Others include cytosine deamidase (cd). For example, the AdV vector encoding tk can he aglatimagene besadenovec. It can be constructed using techniques that are standard in the fields of molecular biology and virology.

Useful prodrugs are those which, when in cleaved form are active in the formation of a molecule which kills the cell. For example, useful anti-herpetic prodrugs include, but are not limited to, ganciclovir, valaciclovir, acyclovir or famciclovir. They can be commercially obtainable from pharmaceutical manufacturers (e.g., Sandoz (Princeton, N.J.), Cipla (Mumbai, India), and Wockhardt (Parsippany, N.J.).

### Combination Therapy

The present disclosure also relates to administration of GMIS therapy according to the disclosure in combination with other therapies that up-regulate TIM-3. Such “combination therapy” may be administered to subjects suffering from or susceptible to increased TIM-3 levels (whether or not resulting from administration of the immune checkpoint inhibitor therapy).

For example, GMIS therapy can be administered in combination with immune checkpoint inhibitor therapy that targets, for example, one or more checkpoint molecules that have been demonstrated to, or are expected to raise TIM-3 levels. Useful checkpoint molecules to target include, but not limited to, PD-1, PD-L1, PD-L2, CTLA-4, CD80, CD86, LAG-3, KIR, TIM-5 and/or Galectin-9. For example PD-1 is an immune checkpoint molecule that down-regulates pathways of T cell activation. PD-1 binds PDL-1 and PD-L2. Blockade of PD-1 PD-L1/PD-L2 interactions augments T-cell activation and proliferation.

Therapy with certain immune checkpoint inhibitors can increase TIM-3 levels in subjects. The present disclosure demonstrates that administration of GMIS therapy in combination with administration of immune checkpoint inhibitors can surprisingly protect subjects against the increase in TIM-3 otherwise observed with the immune checkpoint inhibitor therapy.

Without wishing to be bound by theory, the observed increase in TIM-3 levels during and/or after administration of immune-targeted cancer r therapy may reflect redundancy within the immune checkpoint system. That is, inhibition of one immune checkpoint (e.g., PD-1) may trigger the immune system to elevate another (e.g., TIM-3). GNUS therapy does not trigger TIM-3 elevation.

Alternatively, the observed increase in TIM-3 levels during and/or administration of immune-targeted cancer therapies may reflect an immune inhibitory regulatory function of TIM-3. That is, stimulation of the immune system with immune targeted therapy (e.g., cytokine therapy) may trigger the immune system to elevate immune inhibitory components (e.g., TIM-3), The present disclosure demonstrates that GMIS therapy can protect a subject from TIM-3 elevation that would otherwise result from administration of immune-targeted therapy. The present disclosure documents that improved overall survival rates can be achieved when GMIS therapy and immune checkpoint inhibitor therapy are administered in combination relative to that observed when either therapy is administered alone. The GMIS therapy may trigger cytotoxic tumor lysis, resulting in release of tumor antigen, and that its combination with immune checkpoint inhibitor therapy, when TIM-3 is not elevated (i.e., when even redundant immune checkpoints are suppressed), permits an increase in T-cell activation associated with such release, so that a subject's immune system can more effectively destroy cancer cells.

TIM-3 levels observed upon administration of the combination therapy may be comparable to those observed absent the immune checkpoint inhibitor therapy. In these instances, the TIM-3 levels observed during and/or after administration of the combination therapy are comparable to those observed upon administration of GMIS therapy absent immune checkpoint inhibition therapy.

Other therapies that up regulate Tim-3 include, but are not limited to, cytokine-mediated therapy, (e.g., IL-2, IL-7, IL-15, IL-18, IL-21, OL-27, CM-CSF, FLT-3, Interferon), immune adjuvants known to stimulate immune activations (e.g., Toll like receptor agonists such as CpG or GLA), or tumor-associated antigens including a vaccine that may include replicating, or non-replicating microbial vectors viral or bacterial) encoding such antigens.

### Pharmaceutical Compositions

GMIS therapy and/or additional TIM-3-upregulatory therapies, e.g., immune checkpoint inhibitor therapy is administered in a pharmaceutical composition that also comprises a physiologically acceptable carrier or excipient that does not affect the activity of the therapeutic. The pharmaceutical composition is formulated for a particular mode of administration.

Suitable pharmaceutically acceptable carriers include but are not limited to water, salt solutions (e.g., NaCl). saline, buffered saline, alcohols, glycerol, ethanol, gum arabic, vegetable oils, benzyl alcohols, polyethylene glycols, gelatin, carbohydrates such as lactose, amylose or starch, sugars such as mannitol, sucrose, or others, dextrose, magnesium stearate, talc, silicic acid, viscous paraffin, perfume oil, fatty acid esters, hydroxymethylcellulose, polyvinyl pyrrolidone, etc., as well as combinations thereof. A pharmaceutical preparation can, if desired, comprise one or more auxiliary agents (e.g., lubricants, preservatives, stabilizers, wetting agents, emulsifiers, salts for influencing osmotic pressure, buffers, coloring, flavoring and/or aromatic substances and the like) which do not deleteriously react with the active compounds or interference with their activity. For example, in some embodiments, a water-soluble carrier suitable for intravenous administration may be used.

The pharmaceutical composition containing GMIS and/or the pharmaceutical composition continuing another immune therapy (e.g., anti-checkpoint inhibitor), can contain an amount of wetting or emulsifying agents, and/or of pH buffering agents. A pharmaceutical composition can be a liquid solution, suspension, emulsion, tablet, pill, capsule, sustained release formulation, or powder. A pharmaceutical composition can be formulated as a suppository, with traditional binders and carriers such as triglycerides. Oral formulation can include standard carriers such as pharmaceutical grades of mannitol, lactose, starch, magnesium stearate, polyvinyl pyrrolidone, sodium saccharine, cellulose, magnesium carbonate, etc.

A pharmaceutical composition according to the disclosure can be formulated in accordance with the routine procedures as a pharmaceutical composition adapted for administration to human beings. For example, in some embodiments, a composition for intravenous administration typically is a solution in sterile isotonic aqueous buffer. Where necessary, a composition may also include a solubilizing agent and a local anesthetic to ease pain at the site of the injection. Generally, ingredients are supplied either separately or mixed together in unit dosage form, for example, as a dry lyophilized powder or water free concentrate in a hermetically sealed container such as an ampule or sachet indicating the quantity of active agent. Where a composition is to be administered by infusion, it can be dispensed with an infusion bottle containing sterile pharmaceutical grade water, saline or dextrose/water. Where a composition is administered by injection, (e.g., for injection in to a tumor) an ampule of sterile water for injection or saline can be provided so that the ingredients may be mixed prior to administration.

GMIS therapy and/or another immune therapy can also be formulated in a neutral or salt form. Pharmaceutically acceptable salts include those formed with free amino groups such as those derived from hydrochloric, phosphoric, acetic, oxalic, tartaric acids, etc., and those formed with free carboxyl groups such as those derived from sodium, potassium, ammonium, calcium, ferric hydroxides, isopropylamine, triethylamine, 2-ethylamino ethanol, histidine, procaine, etc.

### Modes of Administration

Pharmaceutical compositions for use in accordance with methods of the present disclosure may be administered by any appropriate route. In some embodiments, a pharmaceutical composition is administered intravenously. The pharmaceutical composition is administered subcutaneously, by direct administration to a target tissue, such as heart or muscle (e.g., intramuscular), or nervous system (e.g., direct injection into the brain; intraventricularly; intrathecally), administered by direct administration to a tumor. Alternatively or additionally, the pharmaceutical composition is administered parenterally, transdermally, or transmucosally (e.g., orally or nasally). More than one route can be used concurrently, if desired.

Various methods may be utilized within the context of the present disclosure in order to directly administer the oligonucleotide (e.g., vector) to the tumor, including direct intralesional injection, intra-cavital, intravenous administration or topical delivery. A lesion may be located, and the vector injected once or several times in several different locations within the body of the tumor. Arteries or blood vessels, which serve a tumor, may be identified and the vector injected into such blood vessel, to deliver the vector directly into the tumor. A tumor that has a necrotic center may be aspirated, and vector injected directly into the empty center of the tumor. A vector may be directly administered to the surface of the tumor, for example, by application of a topical pharmaceutical composition containing the vector. The vector may be administered either directly (e.g., intravenously, intramuscularly, intraperitoneally, subcutaneously, orally, rectally, intraocularly, intranasally, intravesically, during surgical intervention) to the site of a tumor lesion, or be delivered after formulation by various physical methods such as lipofection, direct DNA injection, microprojectile bombardment, liposomes of several types, DNA ligand, administration of nucleic acids alone; or administration of DNA linked to killed adenovirus, via polycation compounds such as polylysine, utilizing receptor specific ligands, as well as with psoralen inactivated viruses such as Sendai or Adenovirus, by electroporation or by pressure-mediated delivery. Vector may be administered by a catheter or tube introduced into the patient.

GMIS therapy and/or an immune checkpoint inhibitor therapy may be administered in a therapeutically effective amount (e.g., a dosage amount and/or according to a dosage regimen that has been shown, when administered to a relevant population, to be sufficient to treat a tumor or cancer, such as by ameliorating symptoms associated with the tumor or cancer, preventing or delaying the recurrence of the tumor or cancer, and/or lessening the severity or frequency of symptoms of a tumor or cancer). Long term clinical benefit is observed after treatment with GMIS therapy and/or an immune checkpoint inhibitor therapy. Those of ordinary skill in the art will appreciate that a dose which will be therapeutically effective for the treatment of tumor or cancer in a given patient may depend, at least to some extent, on the nature and extent of tumor or cancer and can be determined by standard clinical techniques.

For example, the vector may be administered at various doses, e.g., between 10⁴ and 10¹⁵ vector particles (vp). Titers of vector in a subject can range between 10⁸ vp/ml and 10¹³ vp/ml. A patient can be administered a 0.3 ml to 500 ml dose of vector as a single bolus dose, or as a repeated dose. The vector may be administered to a tumor ranging in size from 1 cm to 20 cm. A dose may be administered in a single bolus injection. Alternatively, a dose may be administered as multiple injections within a single tumor site. The multiple injections may be administered at about the same time or over a period of time, for example, over several hours, over several days, over several weeks or over several months. Alternatively, a dose consisting of up to 500 mls per day, can be administered over a time period of 5 days in order to establish one course. Patients can receive as many courses as necessary in order to establish a response without proving toxic. Courses can be given, for example, weekly or every other week over months or over years.

In another example, the vector and the prodrug are administered according to an intermittent dosing regimen comprising at least two cycles.

In addition, the additional immune therapy can be administered according to an intermittent dosing regimen comprising at least 2 cycles, where two or more therapeutic regimens are administered in combination and each by such an intermittent, cycling, regimen, individual doses of different agents may be interdigitated with one another. One or more doses of second agent (an additional immune therapy such as an immune checkpoint inhibitor), may be administered a period of time after a dose of the first regimen. As used herein, the “first regimen” is actually GMIS which is made up of two agents, an oligonucleotide-based cytotoxic immune stimulant and a prodrug). Each dose of the second regimen may be administered a period of time after a dose of the first regimen (the collective doses of the two agents making up the dose of the first regimen). Two or more doses of the first regimen may be administered between at least one pair of doses of the second regimen; two or more doses of the second regimen are administered between at least one pair of doses of the first regimen. Different doses of the same regimen may be separated by a common interval of time, but the interval of time between different doses of the same regimen may vary. Different doses of the different regimens are separated from one another by a common interval of time; in some embodiments, different doses of the different regimens may be separated from one another by different intervals of time.

To give one exemplary protocol for interdigitating two intermittent, cycled dosing regimens for GMIS therapy and immune checkpoint inhibitor therapy, one useful protocol of GMIS includes: 1) a a first dosing period during which a therapeutically effective amount a first regimen (vector and prodrug) is administered to a patient; 2) a first resting period; 3) a second dosing period during which a therapeutically effective amount of a second regimen (additional immune therapeutic) is administered to the patient; and 4) a second resting period.

The first resting period and second resting period may correspond to an identical number of hours or days. Alternatively, the first resting period and second resting period may be different, with either the first resting period being longer than the second one or, vice versa. Each of the resting periods can correspond to about 120 hours, about 96 hours, about 72 hours, about 48 hours, about 24 hours, about 12 hours, about 6 hours, about 30 hours, or 1 hour, or less. The second resting period may be longer than the first resting period, it can be defined as a number of days or weeks rather than hours (for instance about 1 day, about 3 days, about 5 days, about 1 week, about 2, weeks, about 4 weeks or more).

If the first resting period's length is determined by existence or development of a particular biological or therapeutic event (e.g., evidence of tumor cell lysis or reduction in tumor size), then the second resting period's length may be determined on the basis of different factors, separately or in combination (e.g., decreased expression of TIM-3, PD-1, PDL-1, or CTLA-4). Exemplary such factors may include type and/or stage of a cancer against which GMIS therapy (e.g., the first regimen) is administered; identity and/or nature of immune checkpoint protein, identity and/or properties (e.g., pharmacokinetic properties) of the first regimen (e.g., of GMIS therapy), and/or one or more features of the patient's response to therapy with the first regimen. The length of one or both resting periods may be adjusted in light of pharmacokinetic properties (e.g., as assessed via plasma concentration levels) of one or the other of the administered regimens. For example, a relevant resting period may be deemed to be completed with the plasma concentration of the relevant agent within the regimen reaching below about 1 μg/ml, about 0.1 μg/ml, about 0.01 μg/ml or about 0.001 μg/ml, optionally upon evaluation or other consideration of one or more features of the patient's response (e.g., of degree of cancer reduction and/or magnitude and/or type of induced cancer-specific immune response).

The number of cycles for which a particular GMIS anti-checkpoint inhibitor is administered may be determined empirically. Also, the precise regimen followed (e.g., number of doses, spacing of doses (e.g., relative to each other or to another event such as administration of another therapy), amount of doses, etc.) may be different for one or more cycles as compared with one or more other cycles. Ultimately, patient response is paramount.

One or more regimens used in combination in accordance with the present disclosure are administered according to a dosing regimen for which they are approved for individual use. For example, one or more utilized agents or regimens of agents are administered according to a dosing regimen approved by a regulatory authority such as the United States Food and Drug Administration (FDA) and/or the European Medicines Agency (EMEA), e.g., for the relevant indication. However, combination therapy permits another agent or regimen of agents to be administered according to a dosing regimen that involves one or more lower and/or less frequent doses, and/or a reduced number of cycles as compared with that utilized when the agent is administered without provided combination therapy. Alternatively or additionally, an appropriate dosing regimen involves higher and/or more frequent doses, and/or an increased number of cycles as compared with that utilized when the agent or agents are administered other than in the relevant combination therapy.

The following examples provide specific exemplary methods of the invention, and are not to be construed as limiting the invention to their content.

### EXAMPLES

### Example 1

### In Vivo and In Vitro Administration of a Gene Mediated Cytotoxic Immunotherapy (GMCI) for Glioblastoma

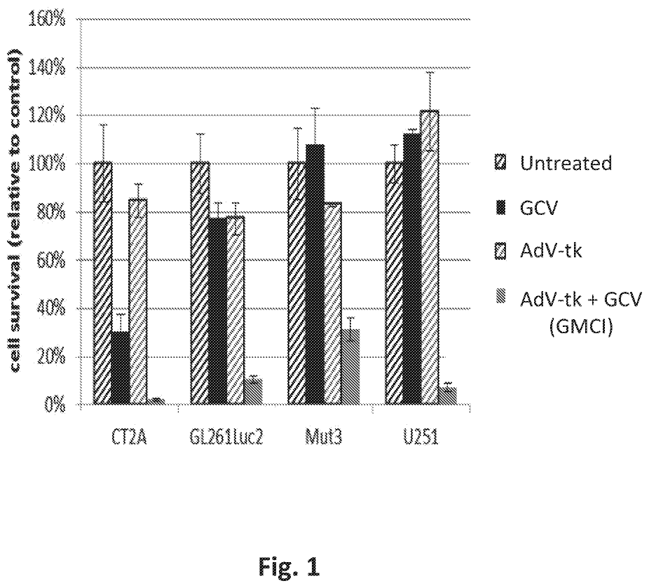

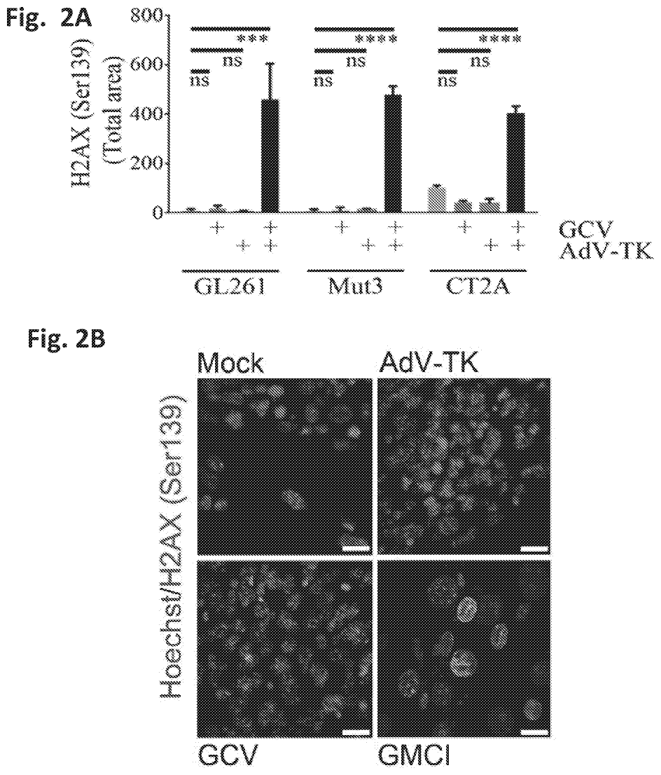

The present Example demonstrates that administration of a GMCI is cytotoxic and causes DNA damage in glioma cells. A mouse model of human high-grade glioma (hHGG) (CT2A), a genetic mouse model of glioma (Mut3), mouse glioma cells (GL261Luc2) and the U251 glioma cell line were treated with control (NC), ganciclovir (GCV or G), an adenovirus-mediated herpes simplex virus thymidine kinase (AdV-tk) or the combination of GCV and AdV-tk (e.g., GMCI). Cell survival was measured relative to control. A significant decrease in cell survival relative to the control, GCV alone and AdV-tk alone was observed in cells and mice treated with both GCV and AdV-tk (e.g., GMCI) (FIG. 1), Immunocytochemical detection of histone H2AX phosphorylated on Ser-139 demonstrated that GL261Luc2 cells, and cells from Mut3 mice and CT2A mice treated with both GCV and an adenovirus-mediated herpes simplex virus thymidine kinase (AdV-tk) have increased DNA damage (e.g., double strand DNA breaks) as compared to cells and mice treated with control, GCV or alone (FIGS. 2A and 2B).

### Example 2

### Increase in Cell Surface PD-L1 in Glioblastoma Cells In Vitro and in Macrophages and Microglia In Vivo with GMCI

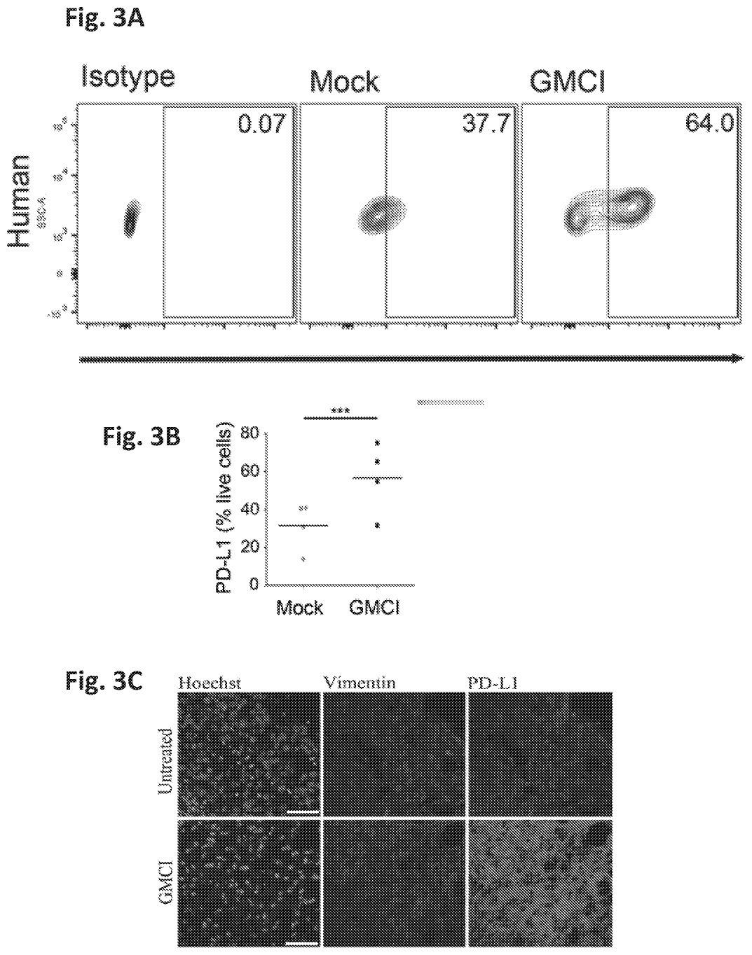

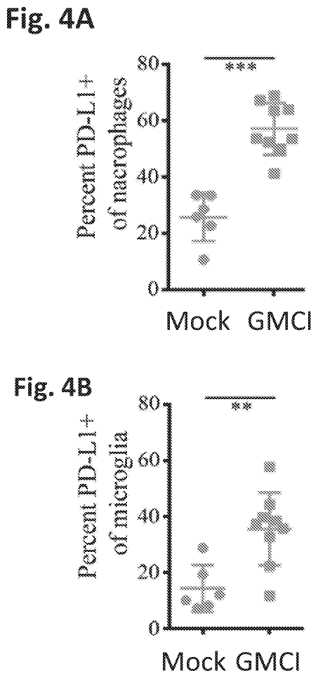

The present Example demonstrates that GMCI treatment of glioblastoma cells increases levels of cell surface PD-L1. Flow cytometry was used to quantify the percentage of cells expressing PD-L1. Cells treated with GMCI demonstrated an increase in the expression of PD-L1 as compared to the expression of PD-L1 on cells receiving the mock treatment (FIGS. 3A and 3B). Immunohistochemical analysis of cells treated with GMCI demonstrated an increase in PD-L1 expression as compared to untreated cells (FIG. 3C). In contrast, GMCI treatment did not increase the expression of vimentin as compared to the expression by untreated cells. Both macrophages (FIG. 4A) and microglial cells (FIG. 4B) treated with GMCI demonstrated an increase in the percentage of PD-L1 positive cells as compared to cells treated with IgG (control).

### Example 3

### Release of INF-β in Mouse Glioma Cells In Vitro

CT-2A and GL261 glioma cells were treated with AdV-tk (10 vp/μl), GCV (10 μg/ml), GMCI (AdV-tk GCV), or were Mock treated. After 4 days, cell supernatant was analyzed for INF-β content by ELISA.

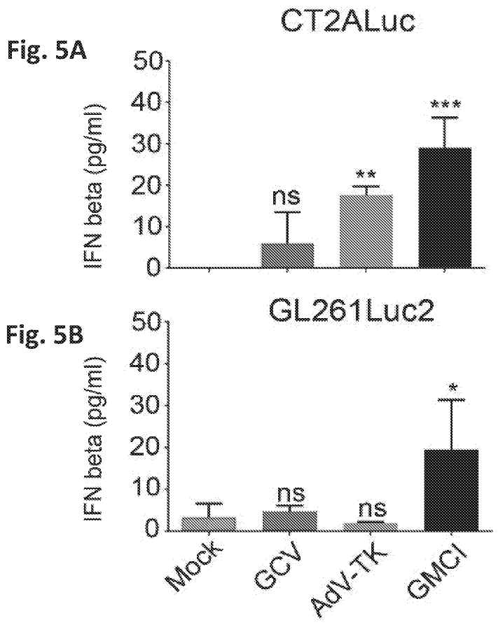

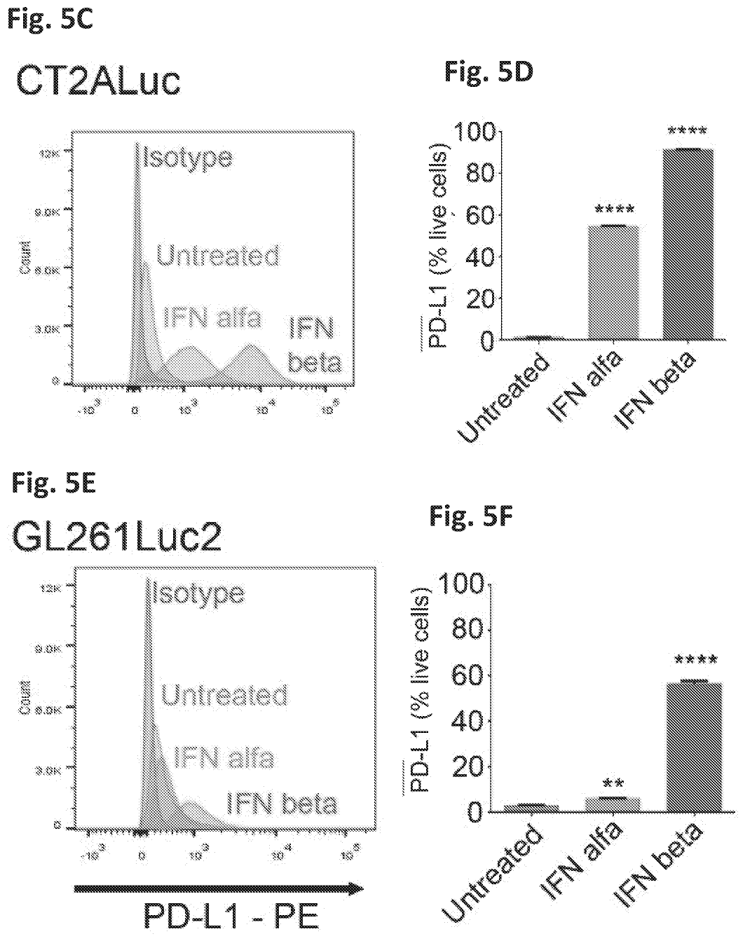

The results demonstrate that treatment of mouse glioma cells CT2A and GL261 vitro with GMCI results in increased release of INF-β as compared to mock treatment (e.g., control), GCV treatment alone or treatment alone (FIGS. 5A and 5B).

In another experiment, CT2A and GL261 cells were treated with IFN-β (1000 U/ml) (PBL Assay Science, Piscataway N.J.) and/or MAR-1-5A3 monoclonal antibody against IFNAR1 (BioXcel, West Lebanon, N.H.) (10 g/ml). Cells were analyzed by flow cytometry for PD-L1 protein expression at day 4 post-treatment using either in the absence of specific antibody (isotype) or with PD-L1 specific antibody (BD Bioscience, San Jose, Calif.). Cells analyzed were either subjected to no treatment (untreated), or treated with INFα or INFβ. Increases in surface PD-L1 expression are correlated with treatment of cell with either INFα or INFβ.

The results in FIG. 5C and FIG. 5E show that in vitro IFN-β treatment of mouse glioma cells CT2A and GL261 in vitro results in increased surface expression of PD-L1 protein as compared to the expression in cell receiving no treatment, mock treatment or treatment with INF-α. FIG. 5D and FIG. 5F show quantification of the percent of live cells analyzed by flow cytometry that express surface PD-L1 above baseline. This indicates that PD-L1 increase observed with GMCI treatment involves INF-β (FIGS. 5C-5F).

### Example 4

### Upregulation of Cyclic GMP-AMP Synthase (cGAS) with GMCI

The expression levels of cGAS in mouse glioblastoma cells were examined after treatment with AdV-TK, GCV and GMCI.

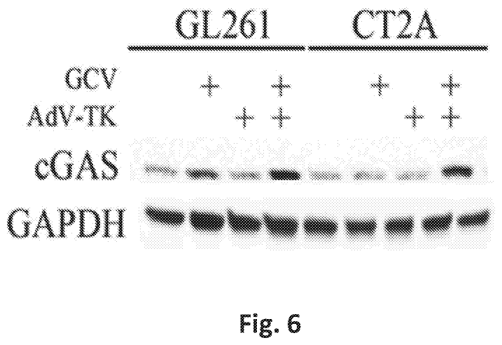

The day following plating of glioma cells, indicated groups were treated with AdV-tk at 10 vp/μl or GCV at 5 μg/ml or AdV-tk and GCV. After 4 days post-infection, cells were harvested and proteins were resolved via SDS-PAGE. Protein expression levels were examined using immunoblot analysis with antibody against cGAS DID3G (15102, Cell Signaling). GAPDH protein expression was detected and used as a loading control. The results are shown in FIG. 6.

This experiment demonstrates that treatment of CL261 or CT2A glioma cells with GCV and AdV-tk results in the activation of the cGAS-STING Stimulator of Interferon Gene (cGAS-STING) pathway, which important in the induction of immune response in tumorigenesis.

### Example 5

### Treatment of Mouse Glioblastoma with GMCI an Anti-PD-1 Antibody

Mice were administered intracranial GL261-Luc2 glioma cells at Day 0. Mice receiving AdV-tk and GCV received intra-tumor (IT) administration of AdV-tk at Day 7 and intraperitoneal (IP) administration of GCV on Days 8 through 17 (e.g., “GMCI:); Mice receiving a-PD1 received IP administration of anti-PD-1 (e.g., “aPD-1”) antibody on Day 10, Day 13, Day 16 and Day 19. Mice receiving combination treatment received intra-tumor (IT) administration of AdV-tk at. Day 7 and intraperitoneal (IP) administration of GCV on Days 8 through 17 and IP administration of anti-PD-1 antibody on Day 10, Day 13, Day 16 and Day 19. The untreated mice did not receive any treatment. Mice were and assessed for survival and tumor burden. Mice reaching at least 100 days of survival were designated “Long Term Survivors” (LTS). LTS mice and age-matched, tumor-naïve mice were administered intracranial GL261-Luc2 glioma cells and assessed for survival and tumor burden;

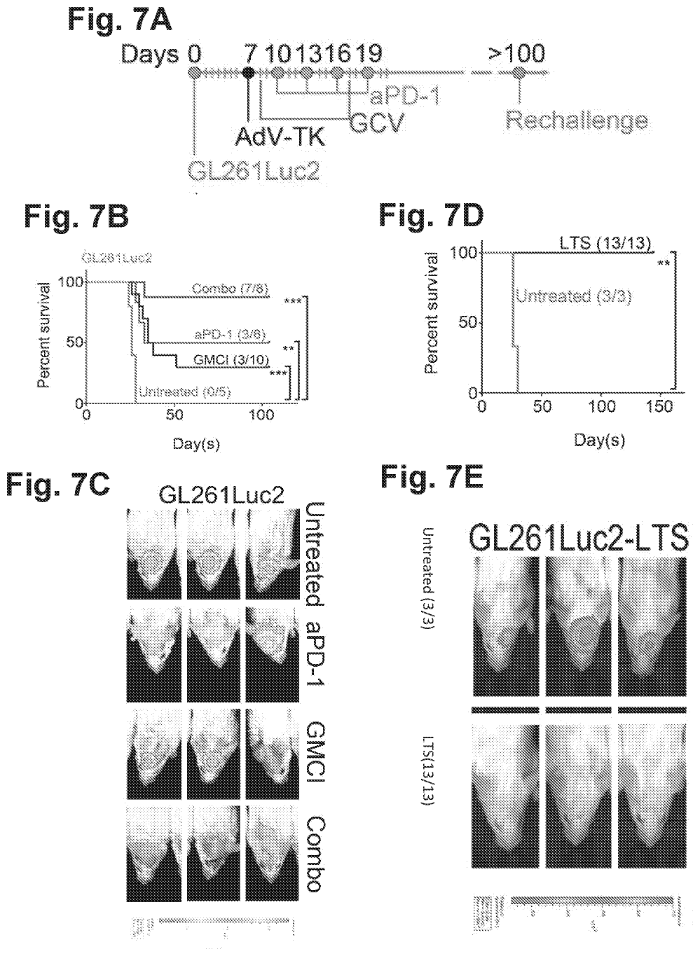

On day 0 mice were administered GL261-Luc2 glioma cells. By day 7, tumors were visualized in the mice. On day 7, the mice received intratumoral (IT) administration of AdV-Tk. On days 8-17, mice received intraperitoneal (IP) administration of GCV. Some mice also received IP administration of an anti-PD-1 antibody on about days 10, 12, 16 and 19. Some mice did not receive either AdV-TK or GCV and were treated with only the anti-PD-1 antibody on about days 10, 13, 16 and 19. Control mice were administered only the GL261-Luc2 glioma cells (FIG. 7A). All control mice died prior to day 50. At day 100, 3 of 10 mice treated with only GMCI (e.g., the combination of AdV-TK and GCV) were alive. At day 100, 3 of 10 mice treated with only the anti-PD-1 antibody were alive. At day 100, 7 of 8 mice treated with GMCI and the anti-PD-1 antibody were alive. The improved survival of the mice correlated with a decrease in tumor burden as observed via bioluminescent imaging (FIG. 7C). The 13 long term survivors (LTS) were rechallenged with GL261-Luc2 cells and all demonstrated long term survival greater than 150 days from day 0 (FIG. 7D). The improved survival of the mice correlated with a decrease in tumor burden as observed via bioluminescent imaging (FIG. 7E).

### Example 6

### Immune Response to GMCI in Combination with Anti-PD-1 Antibody

Tumor-infiltrating lymphocyte populations were prepared from brains on day 21 and analyzed by flow cytometry with results from multiple individual mice being depicted in a scatter plot.

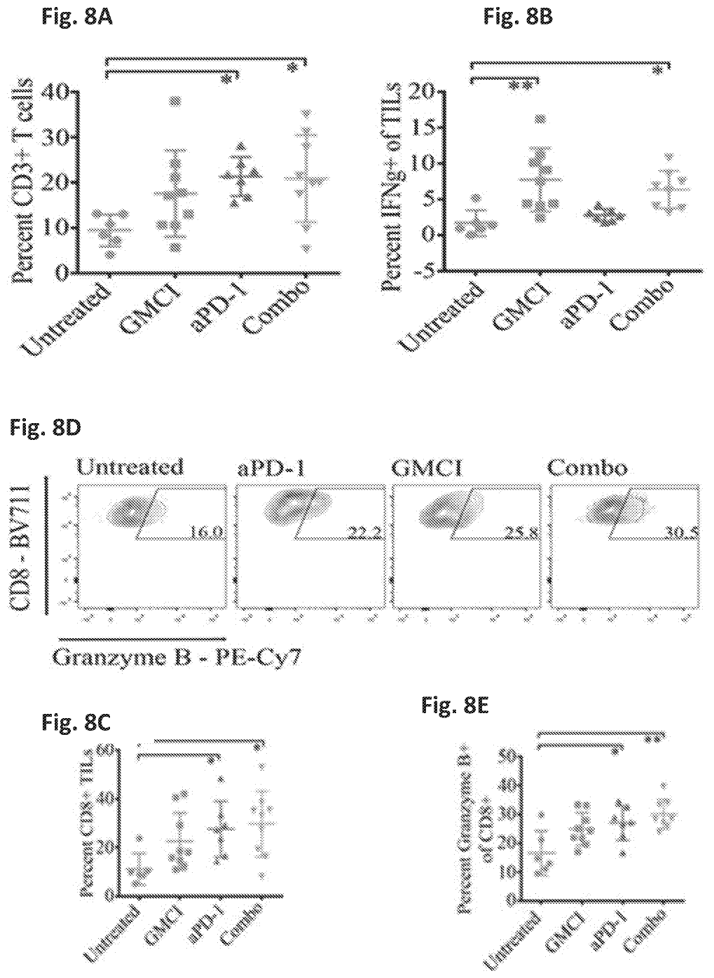

On Day 21 of the protocol illustrated in FIG. 7A, cells were isolated from brains of mice that has been either untreated or treated with GMCI alone, anti-PD-1 antibody alone or with the combination of GMCI and anti-PD-1 antibody. Brains of mice treated with the combination therapy demonstrated a significant increase in the percentage of CD3+ T cells (FIG. 8A), the percentage of INFγ positive tumor infiltrating lymphocytes (TILS) (FIG. 8B), the percentage of CD8+ TILs (FIG. 8C) and the percentage of granzyme B+/CD8+ T cells (FIGS. 8D and E). This Example demonstrates that treatment of mice bearing glioblastoma cells with GMCI in combination with an anti-PD-1 antibody stimulates an immune response.

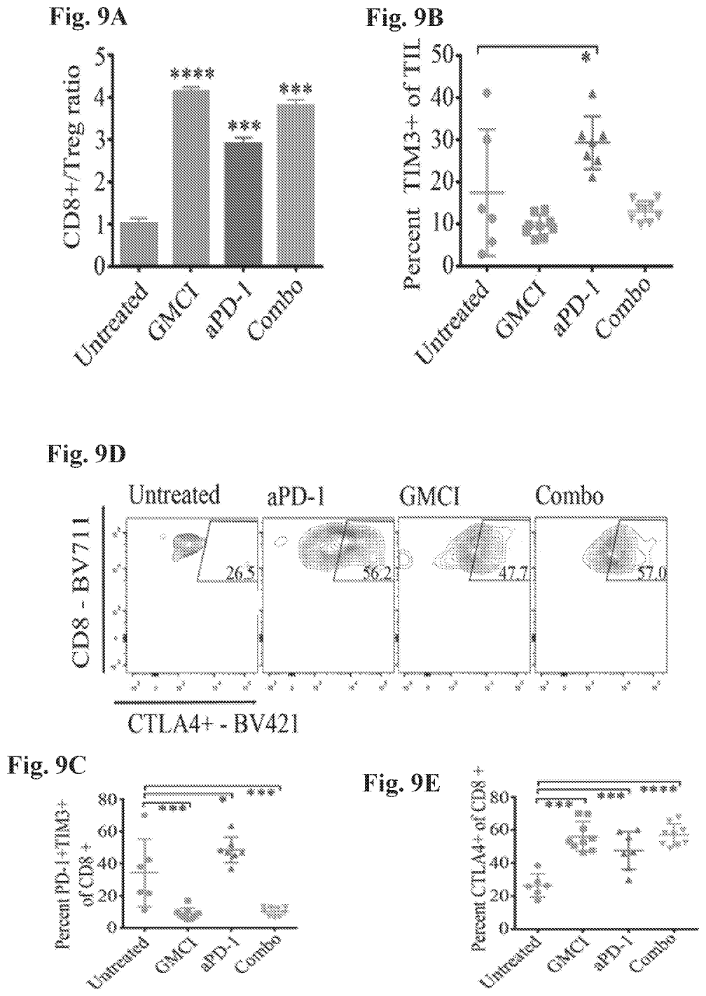

These results also demonstrates that treatment of mice bearing glioma cells with GMCI or anti-PD-1 antibody, alone or in combination, results in a decrease in the CD8+/T_{reg} ratio as compared to untreated cells (FIG. 9A), Tumor-infiltrating lymphocyte populations were prepared from brains on day 21 and analyzed by flow cytometry with the mean results from multiple individual mice being depicted in a bar graph.

TIM3 expression is increased in glioma cells treated with anti-PD-1 antibody alone (FIGS. 9B and 9C). When treatment with the anti-PD-1 antibody is combined with GMCI treatment (“Combo”), TIM3 is down regulated indicating the GMCI is a dominant inhibitor of TIM3. In contrast, treatment with GMCI or anti-PD-1 antibody, either alone or in combination, results in increased expression of CTLA4 in CD8+ T cells (FIGS. 9D and 9E).

### Example 7

### Immune Response to GMCI in Combination with Anti-PD-1 Antibody

Immune cell infiltration levels were characterized in resected tumors of pancreatic cancer patients that had received GMCI treatment and were compared with tissue collected from the patients before treatment. Levels of CD4+ cell infiltrate, or CD8+ cell infiltrate were measured by immunohistochemistry in tissue collected either before treatment with GMCI (“Pre”) or after treatment with GMCI (“Post”). Paraffin sections from pre-treatment biopsy or post-treatment surgical resection for seven patients with available samples were stained with anti-CD4 or anti-CD8 antibody and visualized by secondary antibodies conjugated to fluorophores. The number of positive cells per high-powered field (hpf) counted using microscopic techniques that are standard to those in the field. The count from each of these patients are plotted as the average number of positive cells in three high-powered fields is displayed in the scatter plot for CD4+ cells and CD8+ cells.

The data from the CD4 infiltrate, CD8 infiltrate and PD-LI expression are summarized in a patient-by-patient basis in Table 1.

TABLE 1

| | | | | CD4 | | | CD8 | | |
|---|---|---|---|---|---|---|---|---|---|
| Dose | Patient | PD-L1 | | | | Fold | | | Fold |
| Level | # | Pre | Post | Pre | Post | Change | Pre | Post | Change |
| 1 | 1A03 | − | ++ | 0.67 | 1.67 | 2.49 | 4 | 31.33 | 7.83 |
| 2 | 2A02 | − | ++ | 4 | 9 | 2.25 | 1.67 | 55 | 32.93 |
| 3 | 3A01 | + | + | 1.33 | 0.33 | 0.25 | 1.67 | 125 | 74.85 |
| 3 | 3A02 | + | +++ | 3 | 1.67 | 0.56 | 4.33 | 48.33 | 11.16 |
| 3 | 3A03 | + | +++ | 9.67 | 7.33 | 0.76 | 8 | 98.33 | 12.29 |
| 4 | 4A01 | + | + | 8.33 | 5 | 0.60 | 6 | 39.33 | 6.56 |
| 4 | 4A02 | − | +++ | 3.33 | 7.67 | 2.30 | 6.33 | 38 | 6.00 |

| | |
|---|---|
| Avg. CD4 change: 1.32 | Avg CD8 change: 21.66 |

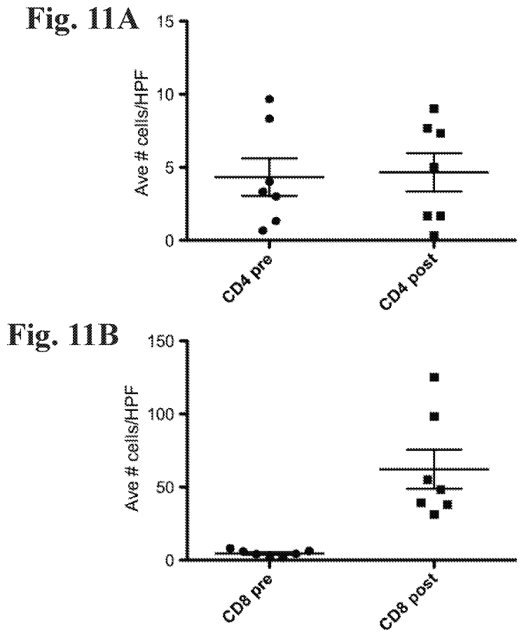

All patients had an increase in CD8+ T cell infiltrate with an average fold increase of 21.66 (FIG. 11B). By comparison, CD4+ T cell infiltrates were not significantly changed (FIG. 11A).

Programmed death ligand (PD-L1) expression levels were also analyzed by immunohistochemistry in tissue collected either before treatment with GMCI (“Pre”) or after treatment with GMCI (“Post”). Paraffin sections from pre-treatment biopsy or post-treatment surgical resection for seven patients with available samples were stained with anti-PD-L1 specific antibody and visualized with a secondary antibody conjugated to a fluorophore. PD-L1 staining was scored using an arbitrary scale by a technician blind to sample type, using standard microscopic techniques.

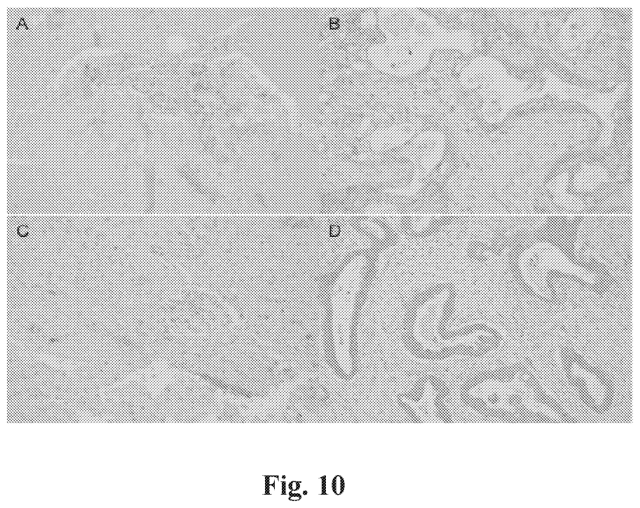

Detection of PD-L1 expression is show to be increase in patient tumor tissue collected after GMCI treatment (FIGS. 10B and 10D) relative to tumor tissue collected in pre-treatment tissue (FIGS. 10A and 10C). These observations indicated that GMCI increases immune activation.

### Equivalents

Those skilled in the art will recognize, or be able to ascertain using no more than routine experimentation, many equivalents to the specific embodiments of the invention described herein. The scope of the present invention intended to be limited to the above Description, but rather is as set forth in the following claims.

We claim:

1. A method of inhibiting TIM-3 mediated down-regulation of immune effector cells in a subject having an immune response to a tumor, comprising:
administering to the subject a therapeutically effective amount of a gene-based cytotoxic immunostimulant (GNUS) therapy effective to up-regulate effector T cells function,
wherein tumor burden in the subject is reduced.

2. The method of claim 1, wherein the GMIS therapy comprises administering an oligonucleotide-based cytotoxic immune stimulant and a prodrug.

3. The method of claim 2, wherein the oligonucleotide-based cytotoxic immune stimulant comprises a virus-based immune stimulant.

4. The method of claim 2, wherein the oligonucleotide-based cytotoxic immune stimulant comprises a gene-based immune stimulant.

5. The method of claim 3, wherein the oligonucleotide-based cytotoxic immune stimulant comprises an adenoviral vector, an adeno-associated viral (AAV) vector, a Herpes viral vector, a vaccinia viral vector, a retroviral vector, or lentiviral vector.

6. The method of claim 5, wherein the oligonucleotide-based cytotoxic immune stimulant comprises an adenovirus-mediated Herpes simplex virus thymidine kinase (AdV-tk) or cytosine deamidase (CD).

7. The method of claim 5, wherein the AdV-tk comprises aglatimagene hesadenovec.

8. The method of claim 6, wherein the prodrug comprises an anti-herpetic pro-drug.

9. The method of claim 8, wherein the anti-herpetic pro-drug comprises ganciclovir, valaciclovir, acyclovir, famciclovir, pemcyclovir, analogs thereof, or a combination thereof.

10. The method of claim 2, wherein the prodrug and the oligonucleotide-based immune stimulant are administered concurrently or serially.

11. The method of claim 10, wherein the prodrug is administered after administration of the oligonucleotide-based cytotoxic immune stimulant.

12. The method of claim 11, wherein the prodrug is administered for at least 1 day after administration of the oligonucleotide-based cytotoxic immune stimulant.

13. The method of claim 10, wherein the prodrug is administered before administration of the oligonucleotide-based cytotoxic immune stimulant.

14. The method of claim 2, wherein the pro-drug is administered orally, intraperitoneally, intrathecally, intravenously, intravitreously, intralesionally, or intrapleurally.

15. The method of claim 6, wherein the AdV-tk is administered intratumorally.

16. The method of claim 1, wherein the subject being treated has also been treated or is being treated with an additional therapy that up-regulates TIM-3 expression.

17. The method of claim 16, wherein the additional therapy comprises immune checkpoint inhibitor therapy, cytokine mediated therapy, treatment with an immune activation-stimulating adjuvant, or treatment with a tumor-associated antigen.

18. The method of claim 17, wherein the additional therapy comprises administration of an immune checkpoint inhibitor.

19. The method of claim 18, wherein the immune checkpoint inhibitor comprises an anti-PD-1inhibitor, an anti-PDL-1 inhibitor, an anti-CTLA-4 inhibitor, or a combination thereof.

20. The method of claim 16, wherein the immune checkpoint inhibitor comprises an antibody.

21. The method of claim 20, wherein the immune checkpoint inhibitor is an anti-PD-1 antibody.

22. The method of claim 21, wherein the anti-PD-1 antibody is pembmlizumab, nivolumab, analogs thereof, or mixtures thereof.

23. The method of claim 20, wherein the checkpoint inhibitor comprises an anti-PDL-1 antibody.

24. The method of claim 23, wherein the anti-PDL-1 antibody is durvalumab, Atezolizumab, Avelumab, analogs thereof, or combinations thereof.

25. The method of claim 20, wherein the immune checkpoint inhibitor comprises an anti-CTLA-4 antibody.

26. The method of claim 25, wherein the anti-CTLA-4 antibody is ipilimumab, tremelimumab, MDX-010, analogs thereof, or a combination thereof.

27. The method of claim 17, wherein the additional therapy comprises a cytokine-mediated therapy.

28. The method of claim 27, wherein the cytokine-mediated therapy comprises administration of a therapeutically effective amount of IL-2, IL-7, IL-12, IL-15, IL-18, IL-21, IL-27, GM-CSF, FLT-3, Interferon, or combinations thereof.

29. The method of claim 17, wherein the additional therapy comprises administration of an immune adjuvant.

30. The method of claim 29, wherein the immune adjuvant comprises a Toll-like Receptor agonist.

31. The method of claim 30, wherein the immune adjuvant comprises CpG or GLA.

32. The method of claim 17, wherein the additional therapy comprises administration of a tumor-associated antigen.

33. The method of claim 32, wherein the tumor-associated antigen is in a vaccine.

34. The method of claim 33, wherein the vaccine comprises a replicating or non-replicating microbial vector which encodes the tumor-associated antigen.

35. The method of claim 34, wherein the vector is a viral or bacterial vector.

36. The method of claim 1, wherein the subject being treated is suffering from, or susceptible to, a cancer.

37. The method of claim 36, wherein the cancer is malignant pleural effusion, lung cancer, mesothelioma, colon cancer, prostate cancer, breast cancer, skin cancer, liver cancer, bone cancer, pancreas cancer, ovary cancer, testis cancer, bladder cancer, kidney cancer, brain cancer, head cancer, or neck cancer.

38. The method of claim 36 wherein the cancer brain cancer

39. The method of claim 1, wherein the immune response in the subject s increased.
